(19) United States
(12) Patent Application Publication
(10) Pub. No.: US 2007/0054406 A1
(43) Pub. Date: Mar. 8, 2007

(54) Meter for rapid analysis of unbound zinc in liquids

(76) Inventor: Christopher J. Frederickson, Galveston, TX (US)

Correspondence Address: Patrick D. Kelly;Attorney for Applicant, #403, 11939 Manchester, St. Louis, MO, 63131, US

(21) Appl. No.: 11/221,167

(22) Filed: Sep. 7, 2005

(51) Int. Cl. G01N 33/20

(52) U.S. Cl. 436/81

(57) ABSTRACT

Concentrations of free (or unbound, exchangeable, etc.) zinc in liquids can be rapidly measured by a metering device. The meter comprises an enclosure and frame, a receptacle that will hold cuvettes or other liquid sample holders, a light source with an activating (excitatory) wavelength, a photodetector to detect fluorescent emissions at a different wavelength, and circuitry for creating signals based on photodetector measurements. The measurements use any of several fluorophore reagents that generate increased fluorescence intensity when bound to available zinc in a liquid. Each fluorophore has its own zinc response traits. This requires a different response curve to be determined and used for each fluorophore, to allow fluorescent measurements using a specific fluorophore to be correlated with zinc concentrations. Stand-alone operations, interactions with computers, calibration cuvettes, and disposable supplies are described.

### FIELD OF THE INVENTION

This invention is in the field of biochemistry and medicine, and relates to a device for rapid and convenient measurement of zinc concentrations in biological fluids, such as blood, cerebrospinal fluid, or cell culture media.

### BACKGROUND OF THE INVENTION

In various fields of biochemistry and medicine, there is a growing recognition that concentrations of zinc in biological liquids are highly important. Of particular importance are concentrations of atoms, ions, or other “species” of zinc that can be referred to by terms such as free, unbound, exchangeable, rapidly exchangeable, available, accessible, chelatable, bindable, labile, etc. All of those terms refer to liquids containing zinc atoms (usually in the form of ions) that are either: (i) unattached to other atoms or molecules, or (ii) “loosely associated” with proteins or other atoms or molecules, in ways that allow the zinc to alternate back and forth between attached and unattached forms in a manner that establishes or seeks equilibrium.

For convenience, these zinc species are referred to in the specification as free zinc, since that is a common term with a generally understood meaning among those skilled in the art. However, in the claims, these zinc species are referred to as exchangeable zinc, to avoid potential confusion or disputes over whether protein-associated or similar zinc is actually “free” zinc (this would be analogous to arguing about whether someone who is married, or who works for a company, is a “free” person).

Extensive information is available on how zinc binds, in a manner that can be described as dynamic, adaptive, and equilibrium-seeking, to various amino acids, proteins, and other biological molecules with varying degrees of strength and tightness (also referred to as affinity, avidity, or other terms). Books that address this subject include Frederickson et al 1984, Mills et al 1989, and Prasad 1994; review articles include Vallee et al 1993, Berg et al 1996, and Frederickson et al 2005.

For the purposes of this invention, it is sufficient to point out that in any biological fluid (and in surface waters, chemical effluents that are discharged to the environment, etc.), three aspects of zinc binding are important:

(1) zinc atoms or ions will bind with varying degrees of tightness (or affinity, avidity, etc.) to various proteins and other molecules;

(2) not all of those binding reactions are “tight”, and under conditions that can be affected by numerous parameters (including temperature, acidity, salinity, etc.), some proteins and other molecules will release zinc, usually in ionic form such as Zn⁺⁺, into the liquid that holds the proteins or other molecules;

(3) the actual concentration of “free” zinc ions in a particular biological liquid, at a certain time and under conditions that may vary over time, is a highly important factor. If free zinc concentrations are outside a suitable range, the zinc can alter and in some cases disrupt or destroy the activities of numerous enzymes and other cellular components.

The importance of item 3 must be emphasized and clearly recognized. Because of two factors, cells evolved over the eons in ways that led to zinc becoming an essential “cofactor” in literally thousands of enzymes. Those two factors can be briefly summarized as follows:

(1) Unlike other metals (such as iron, copper, magnesium, etc.), zinc has no “redox potential”. This means that, unlike other metals, zinc poses no risk of altering the negative or positive charge of biological molecules, through either reduction or oxidation.

(2) A single ion of zinc can bind in a stable and simultaneous manner to four different atoms or molecules. As an example, the finger structures in “zinc finger” proteins are formed when a single ion of zinc binds to a total of four cysteine and/or histidine residues in a protein strand.

Over time, those two traits, acting in combination, caused zinc to become widely adopted and used as a mineral cofactor that stabilizes the three-dimensional shapes and conformations of literally thousands of enzymes.

However, too much zinc can be toxic and even lethal to any type of cell or organism. In addition to binding to the side chains of cysteine and histidine residues, zinc ions (which will be positively-charged, usually as Zn⁺⁺, when present in aqueous solutions at physiological pH levels) will be attracted to, and will bind to or associate with, nearly any atom or molecule that has a localized negative charge. For example, essentially all oxygen or nitrogen atoms in biomolecules have at least one “unshared electron pair”, and any unshared electron pair that is accessible on the surface of a biomolecule will create a localized negative charge, which will tend to attract and bind to zinc ions. As a result, if too much free zinc is present in a biological liquid, it can severely clog up and inhibit the ability of various atoms and molecules to carry out their roles. If the levels of free zinc in a solution continue to increase, this will lead to slight, then substantial, then serious, then toxic, then lethal inhibition of various enzymes and other cellular components.

As a result, zinc, proteins, and cells evolved over the eons in complex ways that established a form of partnership and cooperation. Zinc became an essential cofactor that is necessary for supporting life; and, cells and proteins developed mechanisms that allow them to effectively juggle, balance, and manipulate their supplies of zinc, using various biochemical storage methods (including mechanisms that can function as “buffering” mechanisms) that can place surplus zinc in a location that is analogous to being placed in a bottle that is kept on a shelf in a closet; it is out of the way, and yet it remains accessible and available, in case it becomes needed in the future.

This approach to storing surplus zinc, in non-toxic stored forms, was accomplished mainly through proteins that do not require zinc in order to function, but that will bind to zinc in a non-tight, loosely-associated manner. This allows those proteins (which can be called storage proteins, buffering proteins, etc.) to release their zinc and donate it to other molecules, if and when the need arises. In animals, the most notable storage/buffering proteins are albumen, and alpha-macroglobulin 2, both of which are present in all circulating blood; in addition, the amino acids histidine, cysteine, and glutamate play similar roles. These proteins and amino acids help sustain concentrations of free zinc within the proper ranges, in all tissues and liquids in an animal body.

However, various situations can arise in which free zinc levels can rise (or drop) to potentially harmful surpluses (or deficits). As one example, if a researcher is doing in vitro tests on cells or tissue samples in flasks, petri dishes, or other containers in a laboratory, the researcher will need to use a liquid “cell culture medium” that contains a combination of various nutrients, to allow the cells or tissue to remain viable while being treated and tested. Most cell culture media are artificial mixtures of various known compounds (salts, sugars, etc.), usually supplemented with at least one biological fluid (such as fetal calf serum, FCS, which is widely used when mammalian cells are being cultured). Many researchers (and apparently even some manufacturers) do not realize it, but different cell culture media sold by different suppliers contain concentrations of free zinc that vary widely, over such a broad range that serious questions arise about whether some of those culture media may be disrupting or inhibiting various enzymes, organelles, or other components of the cells that are being treated and tested in vitro.

There is no way to know or even estimate how many times improper or inconsistent concentrations of free zinc, in differing batches or differing types of cell culture media, may have caused or aggravated misleading results in in vitro tests done in laboratories around the world. However, it has become clear that free zinc concentrations, in cell culture media, are likely to be important and in some cases crucial in determining whether a batch of cells, being cultured under artificial conditions in cell culture media, can accurately and reliably emulate the activities of those types of cells under normal in vivo conditions. Therefore, if the manufacturers and users of cell culture media had a simple, rapid, and convenient way to measure free zinc concentrations in cell culture media, they could take proper steps to ensure that such concentrations in any set of tests are within proper and desired ranges.

There also are other reasons why concentrations of “unbound” zinc in biological fluids sometimes need to be measured. One set of reasons involves neurology.

In a mammalian brain, as part of the transmission of nerve signals or impulses between neurons, a number of highly important classes of neurons in the brain release free zinc into the synaptic junctions between signal-transmitting neurons, and signal-receiving neurons. When released into synaptic junctions, zinc affects the activities of neurotransmitter receptors and ion channels on the signal-receiving neurons. After a signal transmission (leading to a “firing” or “depolarization” event) has been completed, the free zinc is rapidly cleared out of the synaptic fluids, by a system that pumps the zinc back inside neurons, so the zinc can be reused again, in subsequent nerve impulses.

The release of zinc into synaptic junctions inside the brain (followed by pumping of the released zinc back into the neurons) is entirely normal, healthy, and essential to proper brain functioning. However, if a major crisis cuts off or seriously impairs the blood or oxygen supply to all or part of the brain (such crises include strokes, cardiac arrests, head trauma, an injury that causes severe blood loss, etc.), the pumping system that normally clears zinc out of the synaptic junctions can run out of energy to drive the pumps. If that happens, excess zinc rapidly accumulates in the fluids in the synaptic junctions that normally handle zinc releases. If that process occurs, the zinc will seriously aggravate a process called “excitotoxicity”, which has become well-known to neurologists. The neurotoxic risks posed by too much zinc are described in articles such as Koh et al 1996, Choi et al 1998, Suh et al 2000, and Frederickson et al 2004 and 2005.

To try to prevent or minimize that type of brain or spinal damage after a crisis, neurology researchers have begun testing various drugs that “chelate” zinc, to determine whether such drugs can reduce brain damage after a stroke or similar crisis. Terms such as “chelate” (and chelator, chelating, etc.) refer to compounds that bind tightly to zinc. This type of tight binding reaction effectively inactivates atoms of zinc, and removes those atoms from the pool of free zinc. The early results of that line of research, described in articles such as Koh et al 1996 and Suh et al 2000, have indicated that if zinc-chelating drugs are used in appropriate dosages, and within certain time limits after a crisis begins, they offer good promise as therapeutic agents that can reduce and minimize brain damage caused by excitotoxic crises such as strokes. However, that research is still in the early stages, and it has become clear to experts that such treatments are sensitive to dosage, timing, and other factors.

Using sampling devices such as thin catheters emplaced in locations such as brain ventricles, small samples of cerebrospinal fluid (CSF) can be obtained from the brain of an injured animal or human, at a series of time intervals (such as every 10 or 15 minutes during early treatment, followed by hourly sampling during the later treatment stages). If researchers or physicians could quickly measure free zinc levels in CSF samples throughout the course of a crisis-response treatment regimen, they could determine how a specific patient is actually responding to dosages of one or more zinc-chelating or zinc-buffering drugs, and they could adjust the selection, dosage levels, and timing of the zinc-chelating or zinc-buffering drugs, in ways that could provide optimal benefits while minimizing unwanted side effects. Accordingly, a metering device that can rapidly analyze and indicate the concentrations of free zinc in samples of cerebrospinal fluid, during a crisis-response regimen that may last for hours or days, could greatly facilitate such research, and could greatly enhance therapeutic treatments as well.

The roles and importance of zinc in the brain are not limited to neurological crises such as strokes. Zinc also may play major roles in some neurodegenerative disorders, including Alzheimer's disease. In particular, zinc is found in high concentrations in beta-amyloid plaques, which are one of the defining traits of Alzheimer's disease. Because of its ability to form crosslinking bonds with cysteine and histidine residues in proteins, zinc may accelerate the growth of beta-amyloid plaques in the brains of people suffering from Alzheimer's disease, and testing in both animals and humans has indicated that treatment with a zinc-chelating drug can slow down or even partially reverse the progression of Alzheimer's disease (e.g., Cherny et al 2001, Bush 2002, Ritchie et al 2003).

Still other problems among certain segments of the population are apparently or potentially related to zinc, leading some scientists to use the term, “zinc dyshomeostasis” (e.g., Cuajungco et al 2003) in affected people. “Homeostasis” refers to a dynamic, adaptive, responsive equilibrium that occurs in healthy animals. In dys-homeostasis, the negative prefix indicates that something has gone wrong with the normal processes.

As one example, a protein called calprotectin, reviewed in articles such as Striz et al 2004, normally functions as part of the “innate” immune system, which can respond rapidly to help keep a microbial infection under control for a couple of days, until a full antibody response is generated by the “adaptive” immune system. Calprotectin is carried by leukocyte cells, and released at the site of an inflammation. It chelates and sequesters any available zinc in the area, to prevent the zinc from being used by microbes that need it to grow and reproduce. This is a very useful function; however, there is evidence that some diseases (such as Crohn's disease, cystic fibrosis, and some types of cancers) may be aggravated by too much calprotectin being released too frequently, leading to serious localized zinc deficiencies. Accordingly, at least one researcher in this field, known to the Inventor herein from private correspondence, is attempting to initiate a search for drug candidates that control and reduce calprotectin activity, in ways that can resolve and prevent zinc deficiencies in localized tissues. A convenient metering device for quickly and accurately measuring the levels of free zinc in bodily or cellular fluids would be of great assistance in such research, and potentially even for home use by patients who are suffering from such diseases.

In addition, lozenges containing zinc are widely used by people suffering from colds. However, clinical trials that test this treatment have produced inconsistent and irreconcilable results, as reviewed by Godfrey 1996 and Marshall 1998. Problems that have clouded and confused the debates over whether zinc lozenges actually help treat colds include: (i) differing rates of ion release by different salts of zinc, which are affected by factors such as acidity in the saliva; and (ii) whether other agents in some lozenges (such as Vitamin C) are chelating and inactivating free zinc ions. A metering device that can reliably determine concentrations of free zinc, in saliva from a person sucking on a lozenge, can address and resolve those questions, in ways that may be able to help lead to treatments that will be accepted and agreed upon by researchers, physicians, public health officials, and the public. Similar tests could also help determine whether zinc salts in genital lubricant formulations can help reduce the risk of infection by sexually transmitted viruses.

All of the research described above could be simplified and made more reliable and useful by the availability of a device that can rapidly and reliably measure free zinc levels, in sampled fluids. However, under the prior art, no such metering device has been available.

One set of obstacles that hindered the previous creation of any such metering device arises from a complicating factor that has been encountered in efforts to develop good, useful, and practical “fluorophore” reagents. Background information is provided below on these types of chemical reagents.

Zinc-Binding “Fluorophore” Reagents

The term “fluorescent” indicates that when a certain molecule or material is “lit up” by incoming light having a certain wavelength, it will respond by emitting an altered form of light at a different wavelength. Because of certain laws of physics (light with a shorter wavelength has higher energy, and energy must be conserved), the “emitted” (fluorescent) light will almost always have a longer wavelength (with a lower energy level) than the “activating” light. The difference between the energy level of the “activating” light, versus the energy level of the emitted fluorescent light, will be absorbed by the material. As an example, it is common to use ultraviolet (UV), near-UV, or blue light to trigger a fluorescent emission that will be shifted to a green, yellow, or other longer wavelength.

In many types of chemical measurements where a change in fluorescence is used to measure the existence or rate of a chemical reaction, the measurement usually arises from a “qualitative” change in fluorescence. The term “qualitative” implies a yes-or-no, on-or-off type of condition, which can avoid problems of having to analyze and interpret “shades of gray”. In other words, in most chemical measurements that use fluorescence, the existence and/or rate of a chemical reaction is indicated by the appearance or non-appearance of a fluorescent color that was not present before the reaction. Therefore, a relatively simple measurement of the intensity of the fluorescent color will provide a reliable indicator of how much of the reaction actually occurred.

However, the fluorescent responses from the reagents described below have a crucially different trait, and the results they generate will render measurements and calculations trickier and more difficult. Instead of creating fluorescent emissions having new fluorescing wavelengths that were not previously present before a zinc-binding reaction occurred, zinc binding reactions involving the reagents discussed herein lead to a somewhat increased intensity, in a fluorescent color that already was being emitted by that same fluorophore reagent, before the reagent was altered by binding to zinc. As a result, conventional types of metering device as known in the art cannot simply correlate a color intensity, with a zinc concentration. Various additional steps must be taken, as described below under the description of the invention.

In addition, another crucially important complicating factor had to be addressed and resolved, before a metering device could be developed that can be relied upon to provide accurate results. This additional complication arises from the fact that most reagents that will bind to zinc, in biological liquids, will also have at least some level of binding affinity for other divalent metals, which includes iron, copper, lead, cadmium, nickel, mercury, etc. While those metals will not be present at significant quantities in some liquids that need to be analyzed, they will be present in other liquids, to a point where the complications they can introduce must be recognized and addressed. Those complicating factors have been addressed and resolved by various methods described below.

With those issues in mind as obstacles that had to be addressed and overcome, attention can now turn to a number of fluorophore reagents that will indeed undergo chemical reactions that cause measurable alterations, when the fluorophore reagents bind to free zinc in a liquid. Prof. Steven Lippard, at the Massachusetts Institute of Technology, has been the lead researcher in that work, and articles describing the reagents that he and his coworkers discovered can be have located easily by searching the Medline database, maintained by the National Library of Medicine, using “Lippard” combined with “zinc”. The assignee company herein, Neurobiotex, works cooperatively with Dr. Lippard, and has licensed the use of those reagents in measuring devices that use those reagents.

For convenience, “fluorophore reagents” are referred to simply as “fluorophores” in the text below.

Some of the zinc-binding fluorophores created by Lippard and his coworkers are referred to as “zinpyr” compounds, since they contain a ring structure derived from pyridine, an aromatic ring that contains a nitrogen atom. Several compounds in the zinpyr class have been given designations such as ZP1, ZP4, and ZP8, since their chemical names are long and complex (for example, the full name of the ZP4 compound is 9-(o-carboxy-phenyl)-2-chloro-5-[2-(bis(2-pyridylmethyl)-aminomethyl)-N-methylaniline]-6-hydroxy-3-xanthanone)). These fluorophores are described in articles such as Burdette et al 2001, Burdette et al 2003, and Chang et al 2004.

These agents do not undergo a qualitative, “on-or-off” change in their fluorescent emission levels, when they bind to free zinc. Instead, they display an increased level of “quantum efficiency” (which can also be referred to by terms such as photon efficiency, photon emission efficiency, emission efficiency, fluorescence efficiency or intensity, etc.). For example, a zinpyr fluorophore preparation might display a quantum efficiency of about 0.3, in the absence of any free zinc. This means that if that particular reagent is activated by a certain quantity or intensity of activation energy at a suitable wavelength (such as 480 nanometers, nm, which is in the visible blue range), it will emit roughly 30% of that same number of photons, but at a different wavelength, such as 530 nm, which is visible as green). By contrast, if a fluorophore binds to free zinc at a level that reaches complete saturation, and is then activated by the same intensity of blue light, its quantum efficiency will increase. Quantum efficiencies of some fluorophores increase to a level of about 0.5, while quantum efficiencies of other such fluorophores can increase to levels as high as about 0.9.

Various fluorophores within the zinpyr class have traits that differ, in various respects. For example, the ZP1 compound is relatively hydrophobic and lipophilic; this limits its solubility in aqueous solutions, but it has a relatively high ability to permeate into and through cell and organelle membranes. By contrast, the ZP4 compound is hydrophilic; it is readily soluble in aqueous solutions, but it has lower levels of permeation through lipid membranes.

Other zinc-selective fluorophores are referred to as “zinspy” compounds, as described in Nolan et al 2004. The letter “s” refers to sulfur, and “zinspy” implies that a sulfur-containing compound (usually in the form of a thioether-pyridyl derivative) was used during synthesis. At least some members of the zinspy class of fluorophores reportedly have improved selectivity for zinc, compared to members of the zinpyr class of compounds; however, zinspy compounds tend to be more water-soluble than various zinpyr compounds, and the reduced lipophilic levels of zinspy compounds reduces their ability to permeate through cellular membranes.

Accordingly, over the past five years, researchers have identified and described various fluorophores that generate fluorescent emissions with increased intensity and efficiency, when they bind to zinc, compared to when they are not bound to zinc. However, major obstacles remained before that type of chemical research could be converted into practical, useful, and inexpensive yet accurate and reliable devices for use in laboratories.

Accordingly, one object of this invention is to disclose an electronic device and system that allows simple, rapid, and convenient measurement of free zinc concentrations, in liquid samples that are loaded into convenient cuvettes or similar holders that can be placed into the metering device.

Another object of this invention is to disclose an electronic device and system that allows simple and convenient measurement of free zinc concentrations, in ways that will nevertheless be accurate and reliable, and that can be controlled in ways that prevent and minimize interference and inaccuracies caused by potentially competing elements, including potentially divalent metals such as copper, iron, nickel, cadmium, mercury, etc.

Another object of this invention is to disclose a system that uses a metering device that uses fluorescent emissions that are altered when fluorophore reagents bind to free zinc, coupled to a computer that can help an operator perform various adjustments and calculations, in a way that will establish a response curve having certain traits and relevance, which can then be used to correlate a measured fluorescence value, from a sample liquid, with a free zinc concentration.

Another object of this invention is to disclose a system for accurately measuring free zinc levels in liquids, involving: (i) a self-contained machine or device that will receive and hold cuvettes or other cup-type holders filled with any liquid that is of interest; (ii) a supply of disposable cuvettes that can be used whenever needed, to measure free zinc concentrations in any liquids that are loaded into the cuvettes; and, (iii) calibration cuvettes, which have been pre-loaded with solutions, films, or other materials that contain known concentrations of zinc, for use in periodically calibrating and adjusting the measuring machine, to ensure high levels of accuracy.

Another object of this invention is to describe second- and third-generation metering devices for measuring free zinc, in which various calculations that are performed by a connected computer, in first-generation metering systems, can be transferred to a microprocessor and display system contained within the metering device.

These and other objects of the invention will become more apparent through the following summary, drawings, and detailed description.

### SUMMARY OF THE INVENTION

A device, system, and method are disclosed for rapid and convenient measurement of concentrations of free (or unbound, exchangeable, etc.) zinc in liquids. The device, referred to herein as a zinc meter (or pZn meter, using a notation comparable to pH or pO₂), comprises the following main components:

a. an enclosure and support component, which typically will be a box-type enclosure with internal frame and attachment components that will support the electronic and other components;

b. a sample-holding component that will accept and hold a device (such as a cuvette, tube, hollow needle, parallel slides, etc.) that holds a sample of a liquid that is being measured, after the liquid has been mixed with a special “fluorophore” reagent that will react with free zinc in a liquid sample;

c. a light source having a first activating (excitatory) wavelength, which will activate fluorescent light emissions (by fluorophore molecules that have reacted with free zinc in the liquid sample being measured) at a different wavelength;

d. a photodetector that can measure the intensity of fluorescent emissions from the liquid sample;

e. electronic means (which can be part of the photodetector, or part of a supporting and interacting circuit) for creating a data signal based on the photodetector measurement, wherein the data signal will be correlated with the intensity of the fluorescent emissions detected by the photodetector (which, in turn, were generated by fluorophore molecules that reacted with free zinc in the liquid being measured); and,

f. means for conveying that data signal to a computer, or to another suitable device or circuit that can process and display numerical values.

The fluorescent measurement, from the photodetector in the meter, will be correlated with a response curve that has previously been created for the fluorophore reagent that is being used during a measurement. Each fluorophore will have its own particular response curve, as described below.

If desired, any number of response curves, for different fluorophores, can be programmed into a computer that interacts with a metering device. If this approach is taken, each time a measurement is made with the meter, the computer can automatically calculate, display, and store information on: (i) the fluorescence intensity that was measured by the photodetector, (ii) the free zinc concentration that was calculated, based on that measurement; (iii) the name, number, or other designation of the specific fluorophore that was used to determine which particular response curve was used to convert a fluorescence measurement into a zinc concentration; and, (iv) the date and time of the measurement, and if desired, the name or initials of the person who made the measurement. The computer can also prominently display the name of the fluorophore reagent that was used, and the software can ask or require the operator to confirm that selection, to ensure that the proper response curve was selected for converting a fluorescent measurement into a calculated free zinc concentration.

Nearly all laboratories, factories, hospitals or clinics, or other facilities that will be using a zinc meter will already have one or more desktop or laptop computers, already present and running. Therefore, the meters disclosed herein will be provided with data input and output options that use “universal serial bus” (USB) connectors and cables, since the USB system has become a worldwide standard for enabling computers to interact with multiple “peripheral” devices. The meter will contain a microprocessor (usually in the form of one or more integrated circuits, which will be part of the electronic system inside the box or shell that encloses the meter). It does not need to contain any numerical or other display panel of its own; however, such a display can be provided if desired.

The complete meter package purchased by a user will contain a set of software that will be loaded onto a computer, to enable the computer to interact with the microprocesor(s) inside the meter. This type of software, usually called “driver” software, occasionally will be updated, such as to include additional response curves that may be created in the future, for new reagents. Current driver software will be posted on a website, for downloading by any registered owner of a zinc meter.

Other enhancements and options are described below.

### BRIEF DESCRIPTION OF THE DRAWINGS

FIG. 1 depicts the arrangement of the main components of a pZn metering device as disclosed herein, showing an enclosure box that holds a light source, a first lightwave filter, a square cuvette for holding a sample of liquid, a second lightwave filter, an electronic photodetector for measuring fluorescent light intensity, an optional digital display panel, an internal microprocessor and memory chip, and a computer interface.

FIG. 2 depicts a system of this invention, comprising a metering device in combination with supply of cuvettes, a supply of at least one fluorophore reagent, a set of sealed calibration cuvettes containing known zinc concentrations, and a supply of an ion-binding reagent that chelates other divalent metals, such as copper, nickel, cadmium, etc.

FIG. 3 is an example of a calibration curve that was plotted for the ZP1 fluorophore reagent, showing pZn values (which are logarithmic) on the horizontal axis, and fluorescence intensity values (in arbitrary units, derived from the number of “counts” measured by a charge-coupled photodetector inside a zinc meter) on the vertical axis.

### DETAILED DESCRIPTION

As summarized above, the main physical component of the invention herein is a metering device that can rapidly measure concentrations of free zinc in samples of liquid (such as blood, cerebrospinal fluid, cell culture media, etc.). This metering device (and a larger system that utilizes this metering device) enables the convenient use of a class of reagents (called fluorophores) that undergo chemical alterations that can be detected, when they bind to zinc atoms or ions.

As discussed in the Background section, fluorophores referred to as “zinpyr” or “zinspy” reagents have been described in articles such as Burdette et al 2001, Burdette et al 2003, and Chang et al 2004, and Nolan et al 2004. These reagents do not undergo a “qualitative” change (i.e., a black-or-white, yes-or-no, on-or-off change) in their fluorescent emissions when they react with zinc. Instead, they undergo a more subtle quantitative shift, from a relatively low “quantum efficiency” value (such as about 0.3, for several such reagents), to a higher value (which usually ranges from about 0.5, up to about 0.9, depending on which particular reagent is involved). These increased quantum efficiency values lead to increased fluorescent emissions, when such reagents react with free zinc in a liquid.

In the fluorophore reagents that are of interest herein, fluorescent quantum efficiency levels become higher in the presence of zinc. If desired, alternate reagents can be developed in which binding to zinc will reduce, rather than increase, fluorescence at one or more wavelengths. However, similar reductions in fluorescent emissions also can be caused by cells, particulates, turbidity or cloudiness, or other components in (or factors that may affect) a liquid sample. Therefore, increased fluorescence offers a generally preferable and more reliable indicator, in the meters disclosed herein, compared to reduced fluorescence.

Each fluorophore will have its own particular traits, both in terms of altered fluorescent intensities when zinc is present, and in other matters, such as water solubility, ability to permeate into cell membranes, selectivity for zinc as compared to copper, iron, or other metals, etc. Those traits vary substantially, among different fluorophores. Therefore, steps must be taken to provide methods and means for ensuring that measurements made with any of various different fluorophore reagents will nevertheless be accurate.

This is accomplished by creating a specific response curve for each specific type of fluorophore reagent that is offered commercially, or otherwise being used or evaluated. Accordingly, response curves are discussed in more detail below.

Regardless of which specific fluorophore is used, a surplus concentration of the fluorophore should be mixed with a limited quantity of free zinc. This will ensure that the number and intensity of bound zinc-reagent complexes that will be created in the sample will de-end on the quantity of free zinc in the liquid, rather than on the quantity of the fluorophore. If the measured intensity of light emitted at the fluorescing wavelength approaches a saturation value, or if there is any other reason to suspect that the free zinc in a certain sample has saturated or nearly saturated the binding capacity of the fluorophore, it is a simple matter for a user to simply dilute the liquid that is being tested. This type of dilution is well known to researchers, and it most commonly uses one or a series of 10× dilutions (1 volume of test liquid is mixed with 9 equal volumes of diluent, such as distilled deionized water). If measurements are being made at extremely low levels (such as picomolar or femtomolar concentrations), a supply of diluent liquid that is reliably known to contain no detectable level of free zinc should be used.

Responsive Curves

Any fluorophore reagent suited for use herein will have both: (1) a “baseline” (or starting, unaltered, unbound, etc.) value for its quantum efficiency (also called fluorescence efficiency, emission efficiency, etc.) when no free zinc is present; and (2) a substantially higher quantum efficiency, when the fluorophore molecules become bound to free zinc ions in a liquid. Therefore, if a surplus of fluorophore reagent is mixed with a liquid sample, the extent of the increase in fluorescent emissions, by the sample-reagent mixture, will be limited and controlled by the amount of free zinc that was available to react with the surplus quantity of reagent. If handled properly, this can provide a reliable indicator of how much free zinc was available, to react with the surplus quantity of reagent in the mixture.

However, it must also be recognized that each different fluorophore reagent will have its own baseline (unbound) quantum efficiency, and its own increased quantum efficiency when it becomes bound to free zinc. Therefore, each different fluorophore reagent will generate its own particular response curve.

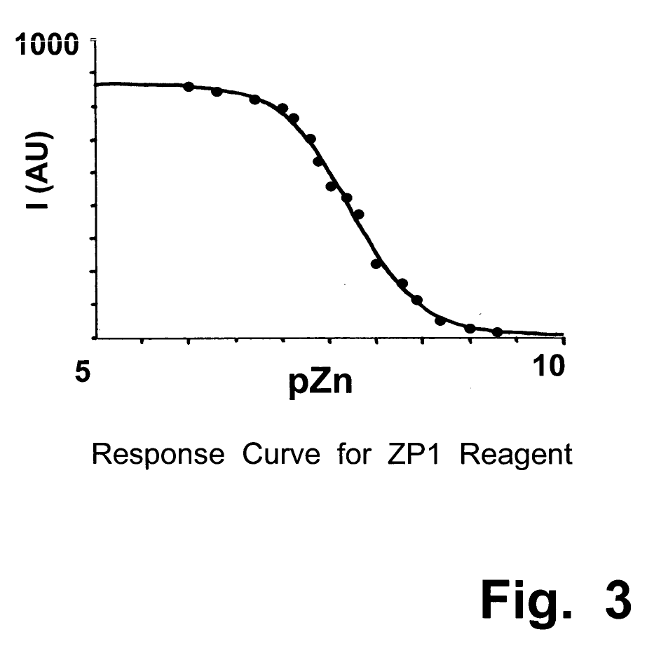

An example of a response curve, for the ZP1 fluorophore, is provided in FIG. 3. Fluorescence intensities are plotted on the Y axis. These numbers will indicate the number of “counts” measured by a photodetector, inside the meter.

Free zinc concentrations are plotted on the horizontal axis. The pZn numbering system is directly comparable to the pH system; i.e., it is a negative logarithmic number, base 10. As examples, a pZn value of 3 indicates a free zinc concentration of 10⁻³, while a pZn value of 6 indicates a free zinc concentration of 10⁻⁶, which is a thousand times lower. Since base 10 logarithmic values are used, concentrations over a very wide range, ranging from femtomolar (i.e., 10⁻⁵), picomolar (10⁻¹²), nanomolar (10⁻⁹), micromolar (10⁻⁶), to millimolar (10⁻³), can be conveniently displayed, using short and convenient numbers, in a manner that is directly comparable to pH values.

Since higher concentrations of zinc will generate greater fluorescent emissions, and since higher concentrations of free zinc are closer to the vertical axis (i.e., higher concentrations correspond to lower pZn values), a response curve such as shown in FIG. 3 will slope downward, toward the right.

Response curves for various different fluorophore reagents are not secret information. Instead, information that can be used to create these types of curves, for various fluorophores, has already been published, in the articles cited above (such as Burdette et al 2001 and 2003, etc.). Alternately, response curves can be readily created for any specific fluorophore reagent that is currently known, or that may discovered in the future, simply by using a meter as disclosed herein to measure test a range of liquids that have been mixed with known concentrations of a zinc salt that dissociates strongly, such as zinc chloride.

If desired, response curves can be estimated by calculations that take baseline versus zinc-bound “quantum efficiency” levels into account. However, at the current time, that is not a preferred approach, and to provide greater reliability, such curves should be created and plotted by actual tests and measurements, using the meters disclosed herein to measure a series of liquid samples having known concentrations of free zinc.

Accordingly, response curves for every fluorophore reagent that is available to anyone using a zinc meter, when a pZn meter is sold, will be included in the literature that accompanies a meter. Such curves also will be posted and readily available, on various websites (including the website of the assignee company herein, www.neurobiotex.com), for any additional and future reagents that may become available in the future from the assignee company.

Once a response curve is available for any particular fluorophore, a meter can be used to accommodate and convert any measurements that are made, using that particular fluorophore reagent, without requiring any physical modifications to the meter.

If desired, any number of different response curves can be programmed into a computer that interacts with a metering device, and the computer can automatically calculate and display both: (i) the fluorescence intensity that was measured by the photodetector, and (ii) the free zinc concentration that was calculated, based on that measurement. on the specific fluorophore reagent that was used. If desired, the computer can prominently display the name of the fluorophore reagent that was used, and it can ask the operator to confirm that selection, to ensure that the proper response curve was selected, for converting a fluorescence intensity measurement into a calculated free zinc concentration. If desired, the supporting software on the computer can also be programmed to record the date and time of each measurement, the initials of the researcher who ran the test, and the serial or other identifying number of the meter. Although that information likely will never be needed, it may become useful, if questions arise at a later date concerning apparent inconsistencies or unexpected observations, especially in research projects involving multiple researchers and technicians.

Under some conditions, the performance of some fluorophores may be affected, to an extent, by the type of liquid being analyzed. Although a specific fluorophore will have generally consistent responses in most types of carrier liquids, differences can arise in how a particular liquid handles activating light and fluorescent emissions, when liquids as different as cerebrospinal fluid versus wastewater effluent are being analyzed. This can become especially important when a liquid contains cells, suspended particles, or other components that may absorb or scatter light at activation or fluorescent wavelengths, or that otherwise create turbidity, opacity, cloudiness, or other factors that influence the behavior or fate of photons passing through the liquid.

Accordingly, response curves using a specific fluorophore in different types of liquid can be created, if a need arises. For liquids that are of widespread interest and that are relatively constant and predictable (such as human blood serum or plasma, cerebrospinal fluid, urine, semen, saliva, sweat, etc.), response curves can be published, posted on a website, or otherwise made available by the assignee company, if the response curve in any particular type of liquid varies significantly from similar curves in other liquids. For other liquids (such as wastewater effluents), response curves can be readily generated by employees, or by contractors or consultants experienced in this type of work, simply by using a pZn meter to test samples of the specific liquid being analyzed, after controlled concentrations of zinc chloride or another suitable salt has been mixed with those liquids.

Computer Interactions; Stand-Alone Meters

If desired, components can be provided to enable partial or even complete stand-alone operation, by a meter that is not connected to a computer. For example, response curves for various fluorophore reagents can be programmed into a microprocessor that has been selected to provide sufficient memory capacity, inside a meter. This type of programmable information preferably should be capable of revision and replacement, by an operator who has proper authority to update the software that is loaded into a meter. This type of approach can allow, for example, raw data indicating the number of counts that are being detected by a photodetector inside the meter, and the corresponding zinc concentration in the liquid that is being measured (based on a known type of fluorophore reagent) to be displayed on a small display panel that can be provided on a meter if desired. The display panel, in conjunction with a knob, button, or other control device on the surface of the meter box, also can allow an operator to scroll through an assortment of different response curves stored in the meter's memory, to select a response curve that corresponds to a particular fluorophore.

However, even if a meter is provided with “stand-alone” capability, it nevertheless should be able to transfer data to a computer, such as by means of a “universal serial bus” (USB) port. USB interfaces (which include inexpensive hubs, routers, and cables) have been designed to provide standardized means for allowing a computer to interact with dozens of peripheral devices. A USB cable also can function as the power cord for a pZn meter, since USB cables can provide 5 volt DC current to drive peripheral devices that do not require substantial power.

The so-called “USB-2 system” is regarded as a type or subset of the USB system. However, because of “backward incompatability” issues that prevent USB-2 devices from being able to transfer data to older computers, pZn zinc meters preferably should be provided with the original class of USB (or USB-1) ports.

Alternately, if desired, a “Firewire” interface, a serial, parallel, or Ethernet cable, a wireless system that uses infrared, radiofrequency, or other emissions, or any other comparable system that is already known or hereafter developed, can be used to transfer data from a zinc meter to a computer, microprocessor, or other device.

The components that create and provide a zinc meter as described herein can also be combined with any other compatible system, within a larger and more complex device. For example, means can be provided for using a single metering device to carry out measurements of two, three, or more parameters that may be of interest in various biological fluids (such as pH and/or pO2 levels, concentrations of various other elements such as iron, copper, sodium, calcium, etc.). Any such measurements can be carried out by using already known or hereafter discovered means and methods, which can be provided by a larger and more complex system that will include and incorporate a zinc-measuring system as described herein.

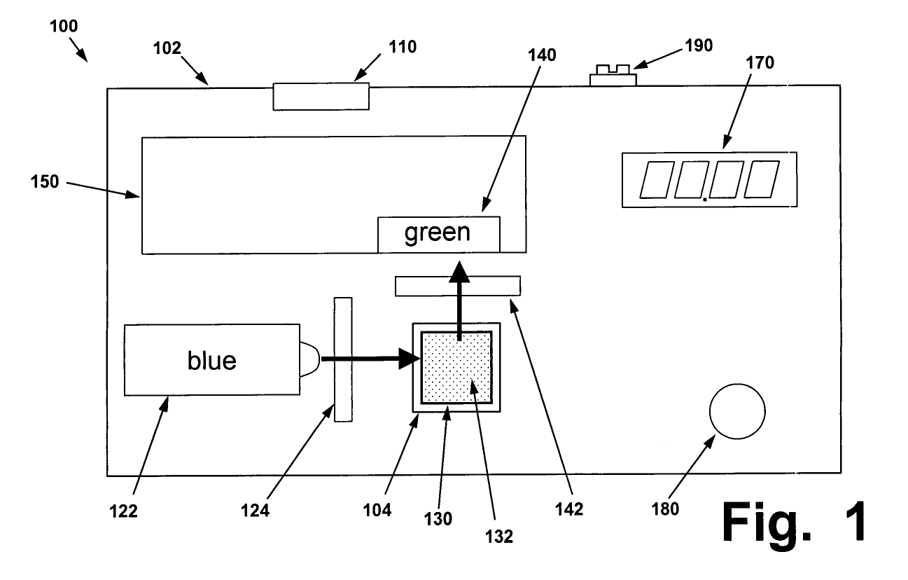

Schematic Depiction in FIG. 1

Accordingly, referring to FIG. 1 as a schematic depiction of an illustrative system for measuring concentrations of free zinc in liquids, callout number 100 indicates a metering device as disclosed herein. Meter 100 is also referred to herein as a pZn meter, wherein the prefix “p” is used in the same way as the “p” prefix in other chemical measurements, such as pH (for acidity), pO2 (for dissolved oxygen concentrations in liquids), etc.

Meter 100 has at least one support and enclosure component 102, which will hold, enclose, and protect various internal components, including several optical components that must be spaced adjacent to each other in controlled locations, as discussed below. Support and enclosure component 102 (which also can be referred to as a box, shell, frame, or similar terms) can be made of one or more pieces of shaped metal, molded plastic, etc. For example, in a preferred mode of construction, an internal frame to which the optical and electronic components can be screwed or bolted (allowing convenient removal and replacement of specific parts) will be made from strips of aluminum, thin-gauge steel, molded plastic, or other suitable material. The outer box or enclosure generally should have a top shell component and a bottom shell component, both of which can be screwed to the frame, allowing either or both of them to be removed, to provide access to the interior components for purposes such as cleaning, repair, replacement, calibration, etc. The meter device preferably also should be provided with rubberized feet or pads on the bottom, to help reduce vibrations or other unwanted motion, and to help protect the unit from spills and chemicals.

Meter 100 must provide a sample-holding component 104 that will allow cup-type sample holders (such as cuvettes) to be easily inserted and removed. “Cuvette” is a conventional term, understood by anyone skilled in this type of research. It is used broadly herein, to include any type of cup-shaped, tube-shaped, or similar device designed to hold a small quantity of a liquid 132 (which can also be called an analyte, sample, specimen, or similar terms). The walls of a cuvette must be made of a material that is transparent to both the excitatory light and the emitted fluorescent light. Most standard cuvettes typically hold about 1 to about 5 milliliters of liquid, and may be etched with a horizontal line to indicate a proper filling depth; however, measurements of very small quantities of liquids can be important, and specialized types of cuvettes or other sample-holders are known that will allow measurements of only a few microliters.

Cuvettes usually are made of a clear plastic (such as polycarbonate, which is relatively resistant to heat, acids, etc.), and they usually are sold and shipped in boxes of 50, 100, or more. They often are regarded as “disposable” supplies; although some cuvettes can be cleaned and reused if desired, there is a risk of scratching, scuffing, or other abrasion if they are cleaned and reused, and there is a risk that some types of chemicals might cling to their internal surfaces if they are not cleaned out thoroughly, with a detergent. Since abrasion and/or clinging residues can lead to inaccurate readings, it is safer and more reliable to simply use them as inexpensive disposable items.

Cuvettes conventionally and preferably have square cross-sectional shapes when seen from above (in a “plan view”, when they are holding a liquid). Square shapes can help ensure more consistent readings than round tubes, by causing an excitatory light to pass in a relatively uniform manner through a liquid sample having a consistent thickness, rather than passing through a thick zone in the middle and thinner zones on the sides (as would occur if circular tubes were used). If desired, a lens can be provided between light source 122 and cuvette 130, to ensure that the excitatory light passes through the cuvette in a direction that is essentially linear and parallel, with little or no “spread”; however, that is not essential, since the cuvettes are relatively thin (their interior widths usually are about half a centimeter), and any spread will occur uniformly in all samples, and therefore will be accommodated by calibrating a meter, using liquids having known zinc concentrations, in cuvettes having sizes identical to the cuvettes used for measuring liquids.

As mentioned above, if a conventional cuvette-holding component is provided in a meter, other types of liquid sample holders (such as hollow needles, parallel slides, etc.) can be adapted to fit into the cuvette holder.

Cuvette holder 104 and fluorescing light detector 140 (discussed below) must be surrounded by opaque materials, to prevent ambient light from reaching those two components in ways that could lead to false readings; therefore, as a matter of convenient design, support and enclosure component 102 preferably should enclose the entire metering apparatus, to protect the electronic and other internal components against spills, dust, breakage, etc., and the entire closure shell can be made of opaque material, such as extruded aluminum or sheet metal, or opaque molded plastic.

Sample holder 104 should be positioned beneath a lid, door, sliding panel, or similar “access means” 106 that can be opened and closed. This will allow convenient insertion and removal of cuvettes or other devices containing liquids, while also keeping ambient light out of the system while a liquid is being measured. The movable access means 106 can use a hinged, sliding, or axle-mounted plate, or any other suitable mechanism; for example, FIG. 1 indicates a cover plate 106 that slides on rails 108. The periphery of the lid or door preferably should be provided with a gasket or seal made of a dark resilient material, such as a foam rubber, piled fabric, flexible cylinders of fabric or rubber, etc.

Electrical power must be supplied to the system. This can be done by a conventional power cord; such conventional power cords can carry either (i) 110 volt AC power (or comparable AC voltage at other voltage levels that may be used in other countries), or (ii) lower voltage AC or DC power, supplied by a transformer that can be plugged in to a wall socket. Alternately, since the device will consume only a relatively small amount of power, low voltage direct current can be supplied by batteries, a USB cable, or similar means (a USB connector or port 110 is shown in FIG. 1). A power cord, USB port, or other electrical supply component can be mounted at any convenient location, such as on either side or the back of the box.

If a USB cable or similar system is used to provide low-voltage power, a capacitor and/or rechargeable battery can be provided as part of the power-handling system inside the meter. The capacitor or battery can be charged up between readings, and can provide most of the power that will run the light source and photodetector during a measurement, so the meter will not impose abrupt drains on the power supply. However, it should also be noted that USB hubs and routers having multiple ports are available, with power supplied by means of a cord plugged directly into a standard outlet. The use of such devices, which eliminate the need for a computer to provide powering voltages to peripherals, can avoid any risk that a sudden power drain by a metering device or other peripheral might cause a computer malfunction.

Meter 100 contains an optical system, which includes at least one light source 122 (such as a bulb with a heated filament, a light-emitting diode, etc.), and at least one light detector 140. It may also contain an optional activation light filter 124 (which can also be called a pass filter, bandwidth filter, or similar terms), if the light from source 122 requires filtering; however, if light source 122 emits only a specific wavelength or narrow bandwidth that does not interfere with measurements of a fluorescing wavelength, activation light filter 124 can be omitted.

As shown in FIG. 1, cuvette 130 is placed directly in the path of the excitatory light. The liquid sample 132 held by cuvette 130 can be pre-mixed with a fluorophore liquid, either before or after loading into the cuvette; alternately, the interior walls of a cuvette may be pre-coated with a fluorophore, before a liquid sample is placed in the cuvette. Pre-coating of fluorophores onto cuvette surfaces may use any of several known techniques, so long as the method chosen is compatible with the needs of the system. For example, cuvette-coated fluorophores may be soluble in certain types of liquids; they may be bonded to porous matrices, gel-type materials, or other materials; or, they may be chemically bonded to cuvette walls, by means of spacer chains having bonding structures that can optimize fluorophore orientation and accessibility.

As indicated in FIG. 1, a light filter 142 with a relatively narrow (or “monochromatic”) bandwidth can be placed between the cuvette holder 104 and the photodetector 140, if desired, to ensure that any emitted light that reaches photodetector 140 will be limited to light within a relatively narrow fluorescing bandwidth. The use of various light filters that can be inserted into and removed from an accommodating slot can allow a variety of different interchangeable light filters to be used in a pZn meter. This approach can allow the use of different fluorophores that may fluoresce at different wavelengths, including fluorophores that have not yet been identified. For illustrative purposes, since several known zinpyr compounds such as ZP1 and ZP4 fluoresce at green wavelengths, FIG. 1 indicates that light filter 142 and photodetector 140 operate at green wavelengths. Alternately, some types of photodetectors will provide fluorescent emission intensity data over an entire spectrum, or over some significant portion of the spectrum. In such devices, correlations of zinc concentrations with fluorescent emission intensity can be based on either: (1) fluorescent intensity at a single specific wavelength, such as 530 nm green emissions when 480 nm blue excitation is used; or, (2) fluorescent intensity over a designated range of wavelengths, such as 520 to 540 nm; the major requirement in such cases is that the response curves for that fluorophore must have been determined, based on the same limits and parameters.

To reduce the incidence of misleading results that may be elevated by unwanted effects, photodetector 140 should be positioned so that it will detect emitted light that leaves cuvette 130 at an angle substantially different from the beam of excitatory light. FIG. 1 indicates a 90-degree “right angle” displacement between the two arrows that indicate the blue excitatory lightbeam and the green fluorescing lightbeam.

Photodetector 140 is wired, soldered, or otherwise electrically connected to an integrated circuit and/or microprocessor assembly 150. Preferably, any integrated circuit or microprocessor 150 should be contained within the measuring device, and it should generate a data signal that can be either (i) transferred to a computer, and/or (ii) converted into a display on a numerical panel that is mounted on the device. However, as will be recognized by those skilled in the art, other circuit and processing systems can be used if desired. For example, photodetector 140 can be selected to merely generate a voltage, resistance, capacitance, or other electronic value, and other components positioned outside the box can interpret that value and convert it into a data signal, numerical concentration, etc.

Integrated circuit or microprocessor 150 can be loaded with any desired combination of either or both of the following: (1) “fixed” instructions that cannot be readily altered by operators; and, (2) programmable capability that will allow the integrated circuit and/or microprocessor 150 to interact with a computer, in ways that an operator can control at will, by using software loaded into the computer.

Such “fixed” instructions can include any combination of (i) algorithms, code, and other instructions that are permanently burned into integrated circuit or microprocessor 150 during manufacture, and (ii) additional “semi-permanent” code, such as code that is often referred to as “electronically programmable read-only memory” (abbreviated as EPROM). In general, EPROM and similar systems are often used to allow only authorized personnel to modify a software set, to reduce the risk that the software might be accidentally deleted, corrupted, etc.). Software that can be revised and updated, but only by qualified suppliers, is common among software programs that allow owners to obtain upgrades, drivers, patches, or other code modifications (collectively referred to herein as “updates”) over the Internet. Typically, these types of software updates are downloaded in the form of an executable file, which will have a command set that will cause the update package to insert the modified lines of code automatically into the prior software, after the update software has been downloaded by a user.

Accordingly, if a zinc meter is coupled (using a USB or similar cable) to a computer that has Internet capability (even if only via a dial-up option), the computer can be provided with a relatively small “update” program that will allow the instruction set in the zinc meter to be periodically updated, as new and improved versions of the software become available over the course of successive years.

Alternately, many types of microprocessors have been developed with proprietary devices that have specialized hardware and access modes to ensure that the software can be revised only by authorized people or companies. These types of proprietary systems often use small plug-in devices, roughly comparable to fuses or circuit breakers but with memory arrays and multi-lead plug-in interfaces, which contain code that can be modified only by using specialized machines. These types of devices and systems are well known to people who specialize in designing, building, and programming “dedicated” microprocessor devices (often referred to by terms such as “programmable logic circuits”).

For zinc meters as disclosed herein, an already-known device called a spectrofluorimeter, available from a company called Ocean Optics (www.oceanoptics.com), can be used to provide a number of the electronic and photodetector components of the machines described herein. Briefly, this type of device is believed to use a diffraction grating or similar device that disperses light into spectrum, which is projected onto a linear “charge-coupled device” array. Ocean Optics also provides software that can be used to interact with those devices; however, more sophisticated software is often be developed for particular uses.

Integrated circuit and/or microprocessor 150 will also be connected (via multi-lead cables or similar means) to the external USB port 110, and to any display devices 170 that may be mounted on the meter. In addition, a control button 180 (or any other suitable switch or activator device) preferably should be wired into the electronic system, in a manner that will trigger a measuring and data-sending operation, each time the button is pressed.

If a pZn numerical system is used, it will display a single number that will automatically accommodate a very wide range of possible values, where the first number in the pZn value indicates the exponent in a base 10 measuring system. As indicated above, this type of numerical system conveniently allows “p” values to accurately indicate any value within a huge range (for example, pH values that range from 1 to 14 can indicate acidity or alkalinity levels covering 14 “orders of magnitude”. Accordingly, only a single display panel 170 would be required for such a system. As mentioned above, display panel 170 can even be eliminated from the meter, and replaced by display of the data on a computer monitor instead.

If desired, integrated circuit and/or microprocessor assembly 150 (or an accompanying computer) also can be provided with memory capability, to allow data from a series of measurements to be stored (any such stored data can also provide the exact time that each such reading was generated, if desired). This can provide backup and support capability, in case data is unexpectedly lost due to a power failure, computer failure, etc.

An adjustment or calibration screw 190 also should be provided on meter 100, either readily accessible on its surface, or protected beneath a covering device if desired. This device can allow periodic adjustment or calibration of the device, using standard solutions having known concentrations of free zinc. Periodic calibration can enable a machine to remain accurate over a span of multiple years, despite factors that may gradually accumulate to a point where they would otherwise begin to affect accuracy (such as, for example, gradual dimming of the light-emitting bulb or diode, accumulation of dust or chemical films on optical surfaces, etc.).

Accordingly, when restated in terms suited for a patent claim, the device or apparatus of the meter itself (without including the cuvettes or other liquid sample holders, or the chemical reagents that will be necessary to operate the meter) comprises:

a. a light source designed to emit an excitatory light at a first known wavelength that can activate a fluorescent emission having a second and different wavelength by a fluorogenic reagent, if the fluorogenic reagent has reacted with free zinc in a liquid that is being measured;

b. at least one component that is designed to hold a liquid sample (such as a receiving well or other fixture for receiving and holding a cuvette, hollow needle, etc.) in the excitatory light pathway, and that allows a liquid sample to be (i) placed in the sample-holding component for a measuring procedure, and (ii) removed from the meter, after the measuring procedure has been completed;

c. a photodetector for measuring the intensity of fluorescent emissions generated by fluorogenic reagent molecules that have reacted with free zinc in a liquid being measured;

d. electronic means for creating a data signal that correlates with the intensity of fluorescent emissions generated by fluorogenic reagent molecules that have reacted with free zinc in a liquid sample being measured, and for conveying the data signal to a display component that can display a numerical value; and,

e. an enclosure and support component that supports the light source, sample-holding component, and photodetector, and that provides a movable enclosure means that allows a liquid sample to be placed in and subsequently removed from the sample-holding component, and that prevents ambient light from reaching the sample-holding component or the photodetector while concentrations of free zinc in a liquid are being measured.

Alternately, this type of device can be also be described and claimed as follows, in language that focuses on the essentials while recognizing that those skilled in the art can assemble these essential components in various ways after evaluating the teachings and disclosures herein:

a. a light source designed to emit an excitatory light;

b. means for holding a liquid sample in a stationary position during a measuring procedure; and,

c. a photodetector capable of measuring fluorescent emissions generated by fluorogenic reagent molecules that have reacted with zinc in a liquid sample being measured; and,

d. an enclosure component that supports the light source, the photodetector, and the means for holding a liquid sample during a measuring procedure, and that is designed to prevent ambient light from reaching the photodetector while a liquid sample is being measured.

Also disclosed and claimed herein is a kit for assembling such a device, containing the components disclosed above, which can be packaged in a way that instructs purchasers or users to assemble the kit into a working device. Clearly, an already-assembled meter that has been tested and checked out by a quality control team is preferable; however, if the claims were limited to pre-assembled meters, infringers might attempt to sell kits or components that would allow purchasers to simply screw together two or more subassemblies, to accomplish the same thing.

Complete Systems: Calibration Cuvettes, Interference Suppressors

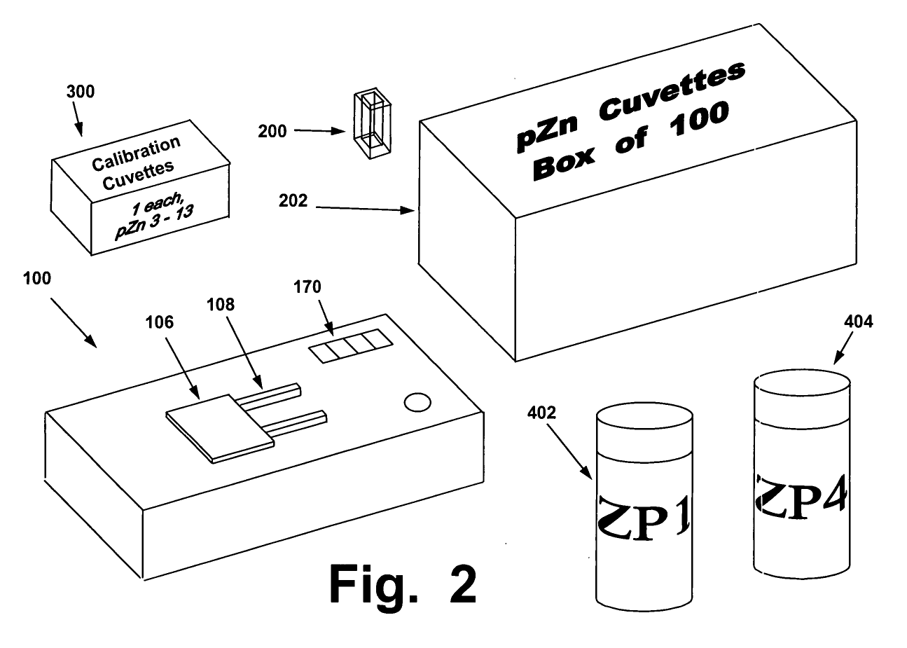

A complete system with additional accessories for measuring free zinc levels is shown in FIG. 2, comprising metering device 100, a supply 202 of cuvettes 200, and a supply of at least one fluorophore reagent, illustrated as bottles 402 and 404 of the “zinpyr” reagents ZP-1 and ZP-4 (unless cuvettes are already coated with such reagents).

Preferably, the complete system also should comprise a set of “calibration cuvettes” 300 that contain controlled mixtures with known concentrations of free zinc. Such calibration cuvettes can be sealed, so that they will not change over time.

In addition, a complete system also can comprise one or more reagents that will bind, strongly and selectively to (or at least with a strong preference for), one or more types of atoms or ions that might otherwise bind and/or react in undesired ways with one or more zinc-binding reagents that are intended for use in a zinc test. In various liquids that may be of interest in different lines of research, transition metals that can be in a divalent (+2) oxidation state under normal conditions (this includes iron, copper, lead, cadmium, mercury, nickel, etc.) can react with some of the zinc-binding fluorophore reagents mentioned above. Depending on the particular fluorophore that is being used, and the types of non-zinc ions that are present, such interference might lead to either: (1) “false positive” binding reactions, which might cause inaccurately high readings, as can occur when cadmium binds to reagents such as ZP1, or (2) “antagonistic” binding reactions (i.e., reactions that will occupy a zinc binding site without triggering a fluorescent change in the fluorophore), which might cause inaccurately low readings, as can occur when copper, mercury, or other elements bind to ZP1.

To prevent these types of unwanted reactions involving other divalent metals, a liquid sample that will be measured can be pretreated by using one or more chelating agents that will bind preferentially to divalent metals other than zinc. As mentioned in the Background section, a “chelating” agent will bind tightly to one or more particular atoms, ions, or compounds, thereby rendering the bound element or molecule unable to react with other compounds. Chelating agents that bind to other divalent metals with much greater affinity than zinc are known, such as certain types of ringed cyclam derivatives, described in articles such as Dong et al 2004.

In addition, there is a long history of quantitative analysis, in chemistry, in which chemists use various agents to measure the concentrations of various metals, in aqueous solutions. To be accurate, most such analytical techniques require an agent to bind tightly to the particular element that is being measured. Accordingly, any agent with a history of use, in quantitative analysis, to measure concentrations of a particular element (such as copper, iron, mercury, etc.) may merit consideration, for use with liquids that contain significant or unusually high concentrations of that particular element.

In addition, certain types of specialized software programs (such as a program called MineQL) and databases (maintained and made available by organizations such as the National Institute for Standards and Technology (NIST) and the International Union of Pure and Applied Chemists (IUPAC)) are available to those who specialize in this type of work. Those software programs and databases can be used to screen and identify candidate compounds that may be useful for sequestering non-zinc metals, prior to measuring the zinc content of a liquid sample.

Most agents that will be identified by one of the approaches mentioned above will bind to one specific and particular element with a high level of selectivity. Accordingly, such agents can be used for analyzing specific liquids that are known to contain particular non-zinc metals, and if desired, mixtures of such agents can be used to create a “cocktail” that will sequester and inactivate two, three, or more such elements, other than zinc.

### METHOD OF THE INVENTION

A method also is disclosed herein for measuring free zinc concentrations in a liquid of interest. This method can be summarized as follows:

a. mixing a known quantity of a sample of a liquid of interest with a reagent that will undergo a chemical alteration when molecules of such reagent react with unbound atoms of zinc to create zinc-reagent complexes, wherein the zinc-reagent displays a measurable physical property that previously was not present in the reagent without zinc;

b. emplacing a liquid-holding device that contains any such zinc-reagent complexes having the measurable physical property into a metering device that: (i) is suited for measuring and quantifying the magnitude and intensity of the measurable physical property generated by the zinc-reagent complexes; and (ii) contains electronic circuitry and processing means for correlating the magnitude and intensity of the measurable physical property generated by the zinc-reagent complexes, with the concentration of zinc-reagent complexes in the liquid-holding device; and,

c. using the electronic circuitry and processing means to generate a data output indicating a concentration of zinc-reagent complexes in the liquid-holding device that was emplaced within the metering device.

In this method, fluorescence is a measurable physical property that is not present in the reagent without zinc, but that becomes present when zinc-reagent complexes are formed; and, a metering device that is suited for measuring and quantifying the magnitude and intensity of such fluorescence can be provided by utilizing an excitatory light source that emits light at an excitatory first wavelength that activates fluorescent light emissions at a fluorescing second wavelength by said zinc-reagent complexes, an a light sensor that detects light at said fluorescing second wavelength.

Thus, there has been shown and described a new and useful device, system, and method for determining concentrations of free zinc in samples of liquid. Although this invention has been exemplified for purposes of illustration and description by reference to certain specific embodiments, it will be apparent to those skilled in the art that various modifications, alterations, and equivalents of the illustrated examples are possible. Any such changes which derive directly from the teachings herein, and which do not depart from the spirit and scope of the invention, are deemed to be covered by this invention.

### REFERENCES

Berg J M et al, “The galvanization of biology: A growing appreciation for the roles of zinc,” Science 1996 271: 1081-1085

Burdette S C et al, “Fluorescent sensors for Zn(2+) based on a fluorescein platform: synthesis, properties and intracellular distribution,” J Am Chem Soc 2001 123: 7831-41

Burdette S C et al, “ZP4, an improved neuronal Zn2+ sensor of the Zinpyr family,” J Am Chem Soc 2003 125: 1778-87

Bush A I, “Metal complexing agents as therapies for Alzheimer's disease,” Neurobiol Aging 2002 23: 1031-8

Chang C J et al, “ZP8, a neuronal zinc sensor with improved dynamic range; imaging zinc in hippocampal slices with two-photon microscopy,” Inorg Chem 2004 43: 6774-9

Cherny R A et al, “Treatment with a copper-zinc chelator markedly and rapidly inhibits beta-amyloid accumulation in Alzheimer's disease transgenic mice,” Neuron 2001 30: 665-76

Choi D W et al, “Zinc and brain injury,” Annu Rev Neurosci 1998 21: 347-375

Cuajungco M P et al, “Zinc takes the center stage: its paradoxical role in Alzheimer's disease,” Brain Res Brain Res Rev 2003 41: 44-56

Dong C J et al, “Three-ring branched cyclam derivatives and their interaction with nickel, copper, zinc and cadmium,” Dalton Trans April 2004 21(8): 1264-1270

Frederickson C J et al, “The neurobiology of zinc in health and disease,” Nat. Rev. Neurosci. 2005 6: 449-462

Frederickson C J et al, “Zinc and excitotoxic brain injury: a new model,” Neuroscientist 2004 10: 18-25

Frederickson C J, “Neurobiology of zinc and zinc-containing neurons,” Int. Rev. Neurobiol 1989 31: 145-238

Frederickson C J, “Synaptically released zinc: physiological functions and pathological effects,” Biometals 2001 14: 353-366

Godfrey J C, “Zinc for treating the common cold: review of all clinical trials since 1984,” Altern Ther Health Med 1996 2: 63-72

Koh J Y et al, “The role of zinc in selective neuronal death after transient global cerebral ischemia,” Science 1996 272: 1013-1016

Marshall S, “Zinc gluconate and the common cold: Review of randomized controlled trials,” Can Fam Physician 1998 44: 1037-42

Mills C F, ed., Zinc in Human Biology (Springer-Verlag, New York, 1989)

Nolan E M et al, “The zinspy family of fluorescent zinc sensors: syntheses and spectroscopic investigations,” Inorg Chem 2004 43: 8310-7

Ritchie C W, et al, “Metal-protein attenuation with clioquinol targeting A-beta amyloid deposition and toxicity in Alzheimer disease,” Arch Neurol 2003 60: 1685-91

Striz I et al, “Calprotectin—a pleiotropic molecule in acute and chronic inflammation,” Physiol Res 2004 53: 245-53

Suh S W et al, “Evidence that synaptically-released zinc contributes to neuronal injury after traumatic brain injury, Brain Res 2000 852: 268-273

Vallee B I et al, “The biochemical basis of zinc physiology,” Physiological Reviews 1993 73: 79-118

1. An apparatus for measuring concentrations of exchangeable zinc in liquids, comprising:
a. a light source designed to emit an excitatory light at a first known wavelength that activates a fluorescent emission having a second and different wavelength by at least one selected fluorogenic reagent that generates a fluorescent response that is altered by exchangeable zinc concentration in a liquid sample;
b. at least one sample-holding component that will hold a sample of liquid in a pathway of the excitatory light, and that allows a liquid sample to be (i) placed in the sample-holding component for a measuring procedure, and (ii) removed from the sample-holding component and from the meter after the measuring procedure has been completed;
c. a photodetector for measuring fluorescent emissions generated by fluorogenic reagent molecules within the liquid sample;
d. electronic means for creating a data signal that correlates with fluorescent emission intensity generated by molecules in the liquid sample, and for conveying the data signal to a display component that can display a numerical value; and,
e. an enclosure and support component that supports the light source, sample-holding component, and photodetector, and that provides a movable enclosure means that allows a liquid sample to be placed in and removed from the sample-holding component, and that prevents ambient light from reaching the sample-holding component or the photodetector while concentrations of exchangeable zinc in a liquid are being measured.

2. The apparatus of claim 1, wherein said excitatory light at a first known wavelength travels in a first pathway, and said fluorescent emission having a second and different wavelength travels to the photodetector in a second nonaligned pathway.

3. The apparatus of claim 1, which is also provided with a numerical display panel that can display a numerical value that correlates with a data signal generated during measurement of a liquid.

4. The apparatus of claim 3, which is also provided with means for conveying said data signal to a computer.

5. The apparatus of claim 1, which is designed to provide electrical power to said light source, and to said electronic means for creating a data signal, from an electrical power source selected from the group consisting of a conventional power cord, batteries, and a voltage provided by a universal serial bus cable.

6. The apparatus of claim 1, which is designed to accommodate and detect fluorescent emissions created by an assortment of different fluorogenic reagents, thereby allowing an operator to select a suitable fluorogenic reagent for measuring a zinc level in a specific type of liquid to be measured.

7. The apparatus of claim 6 which is designed to accommodate and detect fluorescent emissions from at least one member of each of the following classes of fluorogenic reagents: (i) zinpyr reagents that comprise a ring structure derived from pyridine, and (ii) zinspy reagents that comprise a sulfur-containing pyridyl derivative.

8. The apparatus of claim 1 wherein said sample-holding component comprises a receptacle that is designed to accept and hold, in the pathway of the excitatory light, disposable cuvettes that hold samples of liquid.

9. A device for measuring concentrations of exchangeable zinc in liquids, comprising:
a. a light source designed to emit an excitatory light;
b. means for holding a liquid sample during a measuring procedure; and,
c. a photodetector capable of measuring fluorescent emissions generated by fluorogenic reagent molecules that have reacted with zinc in a liquid sample being measured; and,
d. an enclosure component that supports the light source, the photodetector, and the means for holding a liquid sample during a measuring procedure, and that is designed to prevent ambient light from reaching the photodetector while a liquid sample is being measured.

10. The device of claim 9, wherein said excitatory light travels in a first pathway, and wherein fluorescent emissions generated by fluorogenic reagent molecules must travel in a second nonaligned pathway to reach the photodetector.

11. The apparatus of claim 9 which is also provided with a numerical display panel that can display a numerical value that correlates with an electronic signal or value generated by the photodetector during measurement of a liquid.

12. The apparatus of claim 9 which is also provided with means for conveying said data signal to a computer.

13. The apparatus of claim 9, which is designed to accommodate and detect fluorescent emissions created by a variety of different fluorogenic reagents as may be selected by an operator in different tests for measuring different liquids.

14. A kit for assembling a device for measuring concentrations of exchangeable zinc in liquids, comprising the following components:
a. means for supporting and positioning a light source designed to emit an excitatory light;
a. means for holding a liquid sample during a measuring procedure; and,
c. a photodetector capable of measuring fluorescent emissions generated by fluorogenic reagent molecules that have reacted with zinc in a liquid sample being measured; and,
d. an enclosure component designed to support a light source, said means for holding a liquid sample, and said photodetector,
wherein said components are selected and suitable for assembly into a meter for measuring concentrations of exchangeable zinc in liquids.
